(19) United States
(12) Patent Application Publication
(10) Pub. No.: US 2016/0213885 A1
(43) Pub. Date: Jul. 28, 2016

(54) PROTECTION AND SECURING APPARATUS FOR EXTERNALLY PROTRUDING MEDICAL TUBES

(71) Applicant: Jodi Carlson, Mohapac, NY (US)

(72) Inventor: Jodi Carlson, Mohapac, NY (US)

(21) Appl. No.: 14/603,057

(22) Filed: Jan. 22, 2015

(51) Int. Cl. A61M 25/02

(52) U.S. Cl. CPC A61M 25/02; A61M 2025/0253; A61M 2025/026; A61M 2025/0266

(57) ABSTRACT

Embodiments described herein include apparatus for securing and protecting medical tubes, such as a PICC line, which are inserted at an end into a body of a human or other animal. According to one aspect, a medical tube protective sleeve apparatus is provided with a securing strap to secure the apparatus to the body. A first pad has an opening through which the medical tube can be placed. The first pad is connected to the securing strap so that the first pad may be secured to the body, positioned such that the medical tube may be threaded from an insertion point of the body through the opening of the first pad. A second pad is attached to the first pad and may be used to secure onto the first pad to protect the PICC line and exit site from containments. The second pad further includes a securing pocket to allow longer PICC lines to be tucked away/curled-up to avoid being snagged in clothing or other surfaces.

### CROSS-REFERENCE TO RELATED APPLICATION

n/a

### STATEMENT REGARDING FEDERALLY SPONSORED RESEARCH OR DEVELOPMENT

n/a

### FIELD

The present invention relates to a protection and securing medical apparatus, and in particular, to a protective sleeve-like apparatus for externally protruding Peripherally Inserted Central Catheters (PICC) lines and medical tubes, thus minimizing the medical tubes from being caught in the patient's clothing and exposed to contaminants.

### BACKGROUND

A Peripherally Inserted Central Catheter (PICC) is often used by threading the device into a person or animal's large vein to provide intravenous access for a prolonged period. The PICC line or medical tubes, can be for example, a flexible silicone rubber, nitinol, nylon, polyurethane, polyethylene terephthalate (PETE) latex, or thermoplastic elastomers. These PICC lines or medical tubing ensure that nutrition and medication can be effectively inserted in the blood stream. The PICC line terminates on the outside of the body at an egress point, often referred to as an “exit site.” The external body of the PICC line or medical tubing may then be further contained or accessed through a plug or cap. Typically, an adhesive device is placed on the top of the exit site which secures the catheter and helps keep the exit site free from contaminants.

The healthcare industry has employed numerous procedures designed not only to prevent the spread of bacteria and containmants to the exit site, but also to prevent the indwelling medical tubes from being snagged and/or pulled out of the body accidentally. Some of these current procedures include; taping a gauze pad to the exit site, applying wound care dressing, or flexible compression sleeve, i.e., stockinette, or some variation thereof. However these current procedures only offer a short-term solution, and require continuous interference to the medical tubing and exit site. For example, each time a gauze pad is changed, the care provider must remove the tape securing the gauze pad, or unwrap the dressing that may be moist from perspiration or blood accumulation. Because of the current procedures, not only is the chance of contaminant exposure increased, but the chances of the catheter or PICC becoming unsecured increase. Additionally, the current procedures designed to protect the exit site and immediate vicinity, actually increase the chances of pathogen and bacteria exposure.

### SUMMARY OF THE INVENTION

Embodiments described herein include apparatus for securing and protecting medical tubes, such as a PICC line, which are inserted at an end into a body of a human or other animal. According to one aspect, a medical tube protective sleeve apparatus is provided with a securing strap to secure the apparatus to the body. A first pad has an opening through which the medical tube can be placed. The first pad is connected to the securing strap so that the first pad may be secured to the body, positioned such that the medical tube may be threaded from an insertion point of the body through the opening of the first pad.

In accordance with one aspect, some embodiments described herein provide a medical apparatus for securing medical tubes, such as PICC lines, of a type having a flexible elongated tube which can extend to a large vein inside the patient or animal's body. The PICC line or medical tubing may include a closure apparatus such as a cap or plug which allows un-impeded access to the PICC line or tubing for the introduction of fluids and medication.

In another aspect, some embodiments include an apparatus for securing a PICC line or medical tube to a body part. A second piece of flexible material may be affixed to the top of the first material to selectively conceal and secure the PICC line or medical tubes when fastened to the first material to ensure the PICC line or medical tubing does not get tangled or caught on a surface. Further, when the second material is un-fastened to the first material, while remaining affixed to the first material at an edge, convenient access to the PICC line, medical tubing, and exit site is provided. The PICC line securing apparatus further includes a pocket on the inside of the second piece of flexible material designed to allow longer PICC lines to be either circled around, or simply tucked away. The apparatus is applied to the body by at least one flexible strap, which can be tensioned, and, in some embodiments, be attached to and detached from the apparatus. The at least one strap may further include a fastening mechanism such as VELCRO® to fasten the apparatus onto a body part. The at least one strap may further include a layer of non-slip material, such as silicone, which is directly in contact with the body and reduces the occurrence of the straps slipping away from the exit site.

Other aspects, advantages, and novel features of the present invention will become apparent from the following detailed description in conjunction with the drawings.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a front view of the protection and securing apparatus for externally protruding medical tubes in an open position with the cover shown in a fastened position;

FIG. 2 is a front view of the protection and securing apparatus for externally protruding medical tubes in an open position with the cover shown in an un-fastened position

FIG. 3 is a view of the protection and securing apparatus for externally protruding medical tubes in a closed position with the cover shown in an un-fastened position;

FIG. 4 is a front perspective view of the protection and securing apparatus for externally protruding medical tubes in closed position with the cover shown in a un-fastened position; and

FIG. 5 is a front view of the protection and securing apparatus in a closed position with the cover shown in a fastened position.

### DETAILED DESCRIPTION

As used herein, relational terms, such as “first” and “second,” “top” and “bottom,” and the like, may be used solely to distinguish one entity or element from another entity or element without necessarily requiring or implying any physical or logical relationship or order between such entities or elements.

Referring now to the drawings wherein like reference numerals designate identical or corresponding parts throughout the views, there is shown in FIG. 1 one embodiment an apparatus 10 in a first, i.e., open position. Flexible securing straps 12 are affixed to the apparatus 10. The flexible securing straps 12 are designed to wrap around a body part and be tensioned for a tight fit in the proximity of a first pad 13 by strap fasteners 14. The at least one flexible securing strap 12 is composed of an elastic material such as nylon. The at least one flexible strap 12 may be “coated” with non-slip material, such as silicone, to prevent the apparatus from slipping away from the body part. The strap fasteners 14 may be composed of hook and fasteners, or a Velcro® material that ensures the flexible securing straps 12 are securely positioned around a limb of the body and tensioned to the first pad 13. A second pad 15 is affixed to the first pad 13 at an edge, and creates a “flap like” cover to ensure the medical tubes don't get tangled or caught on clothing, as well as cover the exit site. The first pad 13 and second pad 15, including the outside surface of the second pad 16 may be composed of cotton material, polyester-cotton blend material, felt material or a neoprene material.

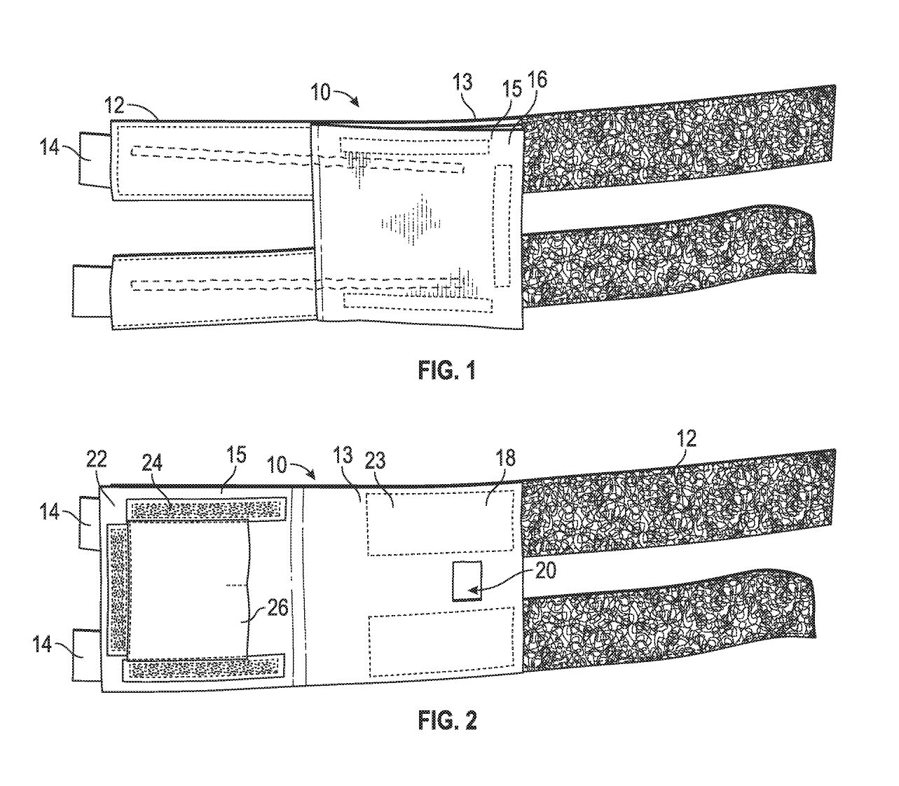

FIG. 2 shows the apparatus 10 in the first position showing the first pad 13 and the outside 18 of the first pad 13 unfastened and exposing the PICC line opening 20 to the caregiver for inspection and disinfecting. The second pad cover 15 may be pulled back exposing the inside 22 of the second pad 15. In order to do this, the user of the apparatus 10 or a caretaker may detach the first pad fasteners 23 from the second pad fasteners 24. The first pad fasteners 23 and second pad fasteners 24 may be made of a hook and loop system, or Velcro® material.

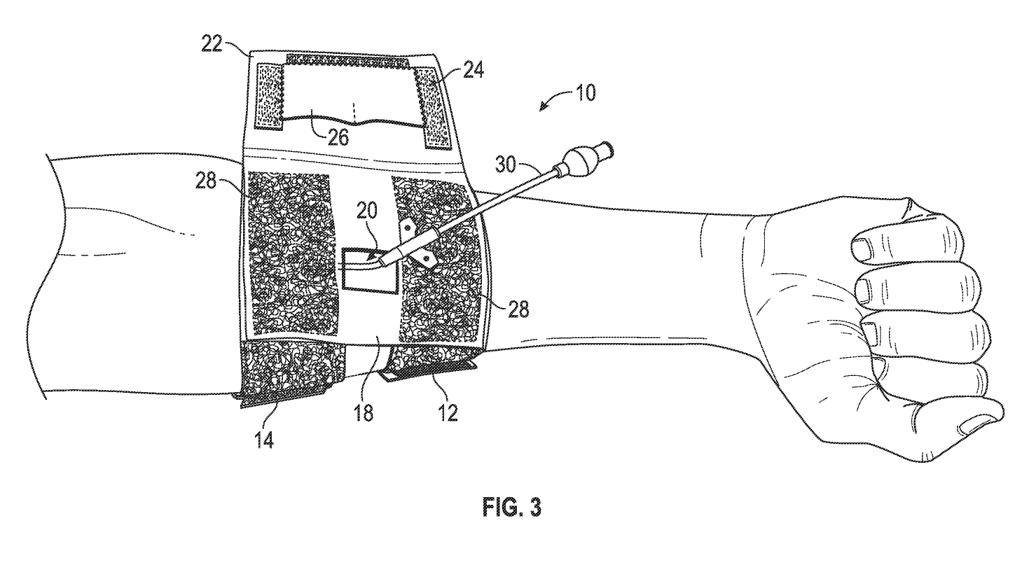

FIG. 3 shows a view of the apparatus 10 attached to a patient's arm in which the apparatus is in a second, i.e., closed position. The second pad cover 15 is unfastened to allow the caregiver access to the PICC line securing straps 26 affixed to the inside cover of the second pad 22, and PICC line 30, threaded through PICC line opening 20. Alternatively, the apparatus may include a storage pocket 26 affixed to inside of the second pad 22.

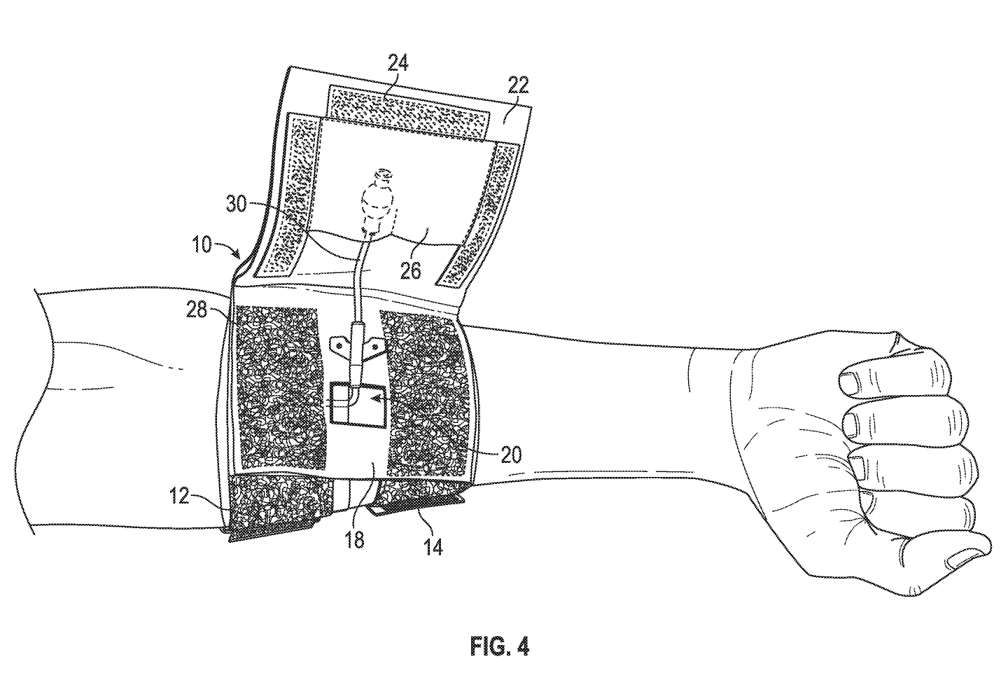

FIG. 4 shows the apparatus 10 secured around a patient's arm in the second position in which the second cover pad 15 may be folded over so that the first pad fasteners 23 affixed to the outside 18 of the first pad 13 will mate with the second pad fasteners 24. The PICC line 30 is threaded through the PICC line opening 20 and then further secured to the inside 22 of the second pad 15 using the second PICC line securing pocket 26.

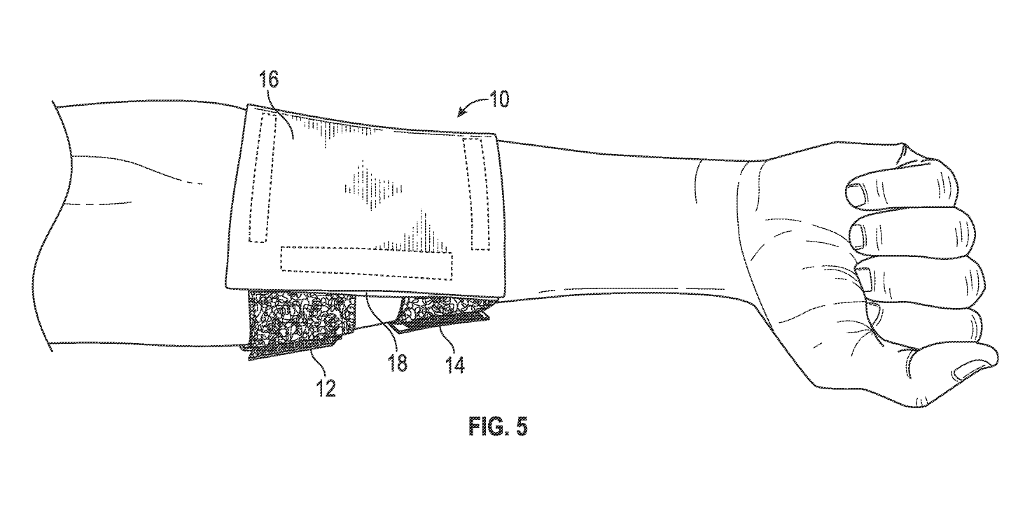

FIG. 5 shows the apparatus 10 secured around the patients arm in the second position with the second pad cover 15 attached to the outside 18 of the first pad 13.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the following claims.

What is claimed is:

1. A medical tube protective sleeve apparatus, comprising:
at least one first securing mechanism to secure the apparatus to a portion of a body of a human or animal, the portion receiving an inserted medical tube;
a first pad having an opening through which the medical tube can be placed, the first pad being connected to the at least one securing part so that the first pad may be secured to a plurality of body portions, the first pad being positioned to allow the medical tube to be threaded from the insertion point of the body through the opening of the first pad.

2. The apparatus of claim 1, further comprising a second pad affixed to the first pad and foldable over the first pad so that when the second pad is folded over the first pad, a opening and a least a substantial portion of the medical tube is covered by the second pad.

3. The apparatus of claim 2, wherein at least one of the first pad and the second pad have fastening mechanisms to removably fasten a surface of the first pad to a surface of the second pad.

4. The apparatus of claim 2, wherein the second pad has a second securing mechanism to secure the medical tube to the second pad.

5. The apparatus of 4, wherein a second securing mechanism is positioned such that when the second pad is folded over the first pad, the medical tube is secured between the first pad and second pad.

6. The apparatus of claim 1, wherein the at least one first securing mechanism is a strap having a fastening material to tension and de-tension the first pad against the portion of the body.

7. The apparatus of claim 2, wherein the at least one first securing mechanism is a strap having a fastening material and a non-slip surface against a portion of the body.

8. The apparatus of claim 2, wherein the fastening material is at least one of Velcro, adhesive, elastic, snaps, or hook and loop.

9. The apparatus of claim 1, wherein the at least one first securing portion is configurable to secure the first pad to the body.

10. The apparatus of claim 1, wherein the first pad is composed of at least one of a cotton material, a polyester-cotton blend material, a felt material, or neoprene.

11. The apparatus of claim 1, wherein the second pad is composed of at least one a cotton material, a polyester-cotton blend material, a felt material, or neoprene.

12. The apparatus of claim 1, wherein the opening is composed of a at least one of a felt material, neoprene, open-cell, or mole skin material around the periphery of the opening.

13. The apparatus of claim 11, wherein the material is adhered to the periphery using an adhesive or elastic material.
